(19) United States
(12) Patent Application Publication
(10) Pub. No.: US 2019/0083056 A1
(43) Pub. Date: Mar. 21, 2019

(54) A SYSTEM FOR CONVERTING A PASSIVE STETHOSCOPE INTO A WIRELESS AND TUBELESS STETHOSCOPE

(71) Applicant: Arash Abiri, Irvine, CA (US)

(72) Inventor: Arash Abiri, Irvine, CA (US)

(21) Appl. No.: 16/087,664

(22) PCT Filed: Mar. 24, 2017
(86) PCT No.: PCT/US17/23929

§ 371 Date: Sep. 24, 2018

Related U.S. Application Data
(60) Provisional application No. 62/312,698, filed on Mar. 24, 2016.

(51) Int. Cl. A61B 7/04; A61B 5/00; H04R 1/46

(52) U.S. Cl. CPC A61B 7/04; A61B 5/0004; A61B 5/742; H04R 2420/07; A61B 2560/0214; A61B 2562/242; H04R 1/46

(57) ABSTRACT

The present invention provides a method to convert any passive stethoscope into a wireless and tubeless stethoscope. A device is mounted onto an exit port on the stethoscope chest piece. The device transduces the acoustic signals from the chest piece into electrical signals, and then wirelessly transmits the signals to a second location to be amplified, filtered, recorded, and played back for the operator with improved sound quality and level. The elimination of tubing further decreases infection risk, increases mobility for the operator, provides a means for telemedicine, and extends access to patient health information and history through electronic medical records.

### CROSS REFERENCE TO RELATED APPLICATIONS

This current application claims priority to U.S. Provisional Patent Application No. 62/312,698 filed 24 Mar. 2016, the disclosure of which is incorporated herein in its entirety by reference.

### BACKGROUND OF THE INVENTION

### 1. Field of the Invention

The present invention relates to stethoscopes and a technique in which stethoscopes can be enhanced to improve upon their limitations.

### 2. Background

Well-known forms of stethoscopes consist of an auscultation chest piece with a diaphragm and/or bell. The diaphragm and/or bell transmit(s) sounds produced by the body through a stem into flexible tubing and then into an earpiece. Despite their global popularity, these acoustic stethoscopes suffer from low sound levels. Furthermore, these stethoscopes come in contact with many regions on multiple patients' body parts such as the neck, chest, abdomen, inguinal area near the pubic region, legs, and feet. The lack of sanitation in stethoscope usage can lead to increased spread of infection from one patient to another. Hospital-acquired infections are detrimental to the healthcare system due to the presence of antibiotic-resistant bacteria and expensive recovery treatments for patients. Although various mechanisms have been developed for improving stethoscope hygiene, the difficulty in isolating and/or cleaning the stethoscope chest piece and tubing persists. The maintenance of hygiene on the long plastic or rubber tubing is particularly troublesome.

Various products have been developed to remedy the low audio level issue of traditional stethoscopes. These products include electronic stethoscopes that amplify and transduce acoustic signals from the chest piece into digital auditory data. However, these stethoscopes have failed to gain popularity among health professionals due to their consequential amplification of noise as well as prohibitively high cost. Stethoscope add-ons have also been introduced. These add-ons perform some of the functions of electronic stethoscopes such as amplification and signal modification as well as recording and playback of the audio. These add-ons consist of a chamber with the electronic components (microphone that picks up an audio signal, electronic circuitry that modifies said signal, and a speaker that plays back the modified signal into the tube) that attaches between the stem of the earpiece and the tubing of the stethoscope. Despite being simple add-on(s) and allowing the physician to keep the original stethoscope, the complexity of the internal circuitry in these devices still drives up device cost. Furthermore, the continued presence of an attached tube means that the serious problem of maintaining stethoscope hygiene persists. Notably, although these electronic stethoscopes are capable of transmitting audio data wirelessly to a remote device, they are still unable to play the audio back in real-time, hence their continued need for a physically connected speaker and tubing for the transfer of audio to an earpiece.

### 3. Definitions

From this point forward in this document, each of the following terms has the meaning associated with it as defined below:
Source is defined as the first and only origin of sound in the system.
Sink is defined as the final and only destination of sound in the system—in all scenarios hereafter, the sink is the auditory organs (i.e. the ears) of the device's human operator.
Physical connection is defined as a connection in which solid matter is present.

Processing is defined as at least one of amplification, filtering, and any form of modification of the audio sample.

Passive is defined as a component that does not use electricity to perform its function.

### BRIEF DESCRIPTION OF THE SEVERAL VIEWS OF THE DRAWINGS

FIG. 1 is an illustration depicting the layout of a stethoscope stem, housing, audio transmission channels, and electronic internal components

FIG. 2 is an illustration of the preferred embodiment of the attachment system between the device housing and stethoscope stem

FIG. 3 is an illustration of another embodiment of an attachment system between the device housing and stethoscope stem

FIG. 4 is a flow chart illustrating the methodology of sound collection, transmission, storage, and display in the device's preferred embodiment

FIG. 5 is an example illustration of a sterilization container

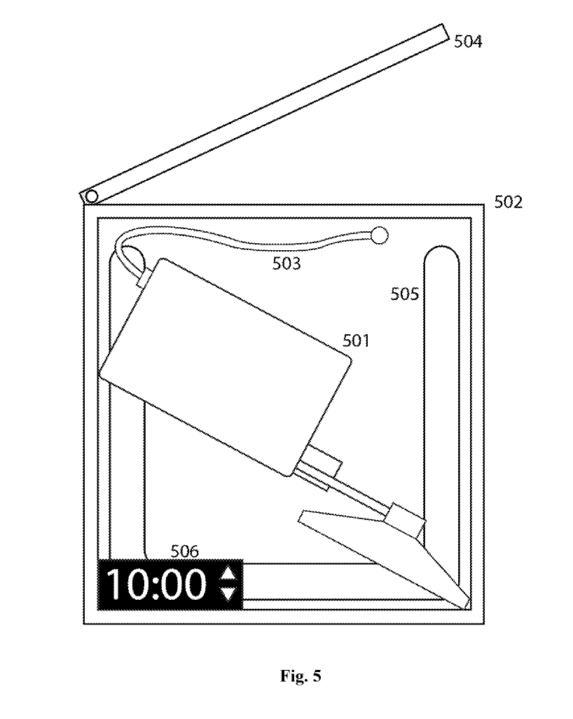

### DETAILED DESCRIPTION OF THE INVENTION

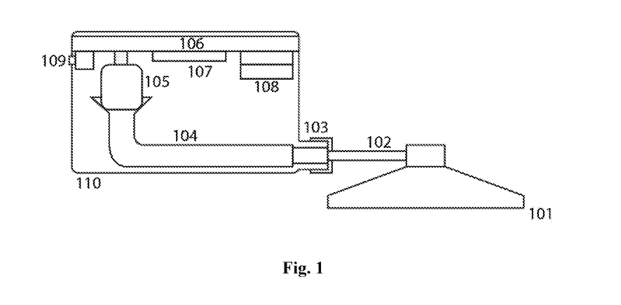

FIG. 1 shows the preferred embodiment of the invention coupled to a stethoscope chest piece. The stethoscope head (101) is intended to be placed in contact with the human body and used to assess audible physiological functions such as heartbeat, breathing, etc. The stethoscope head can be from any manufacturer, as long as the tubing is removable from the stem (102). Even in the case of dual-channel stethoscope stems, the invention can include a plug to seal the stem that is not being used. When in contact with a patient, sound will travel from the stethoscope chest piece, up into the bore of the stem, and towards the body of the invention.

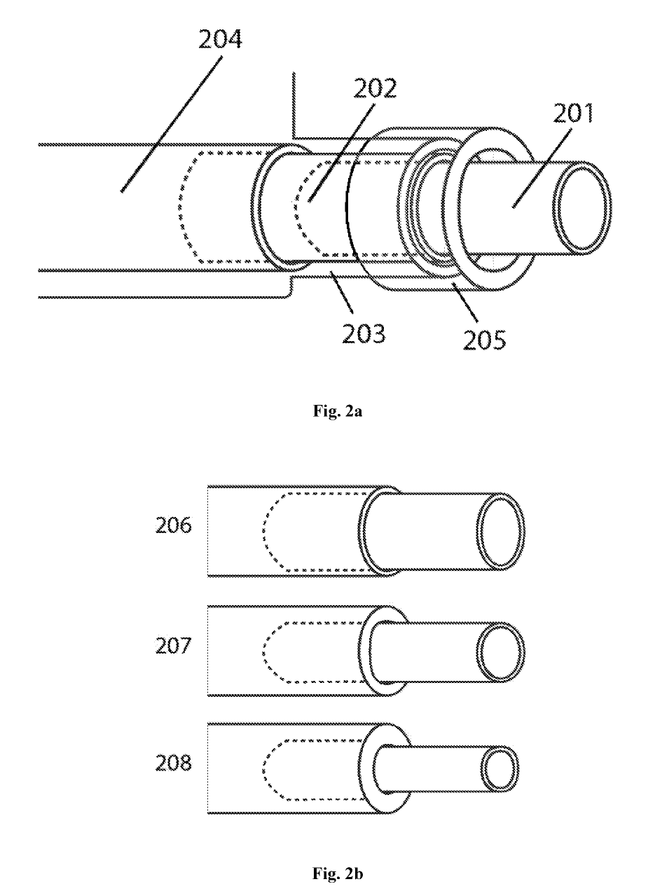

FIG. 2a shows the preferred embodiment of the adapter (103), which connects the stethoscope stem to the body of the invention. The stethoscope stem (201) is inserted into a modular sleeve (202), which is then inserted into a cylindrical protrusion of the device housing (203) in order to provide mechanical support. The modular sleeve has an outer diameter such that it interfaces snugly with the housing protrusion and the internal audio channel (204). The modular sleeve is fastened in place with a cap (205), which in the preferred embodiment is tightened onto the housing protrusion via a helical screw threading mechanism. The cap has a distal inner diameter such that the stethoscope stem can be withdrawn, but the modular sleeve is held inside the housing protrusion. Since the valve component in the preferred embodiment is intended to interface with a variety of third-party stethoscope stems, the modular sleeve will be available in a variety of sizes (FIG. 2b). The outer diameter of all sleeve components is the same, but they have various inner diameters to accommodate stethoscope stems of narrow (206), intermediate (207), and large (208) geometries.

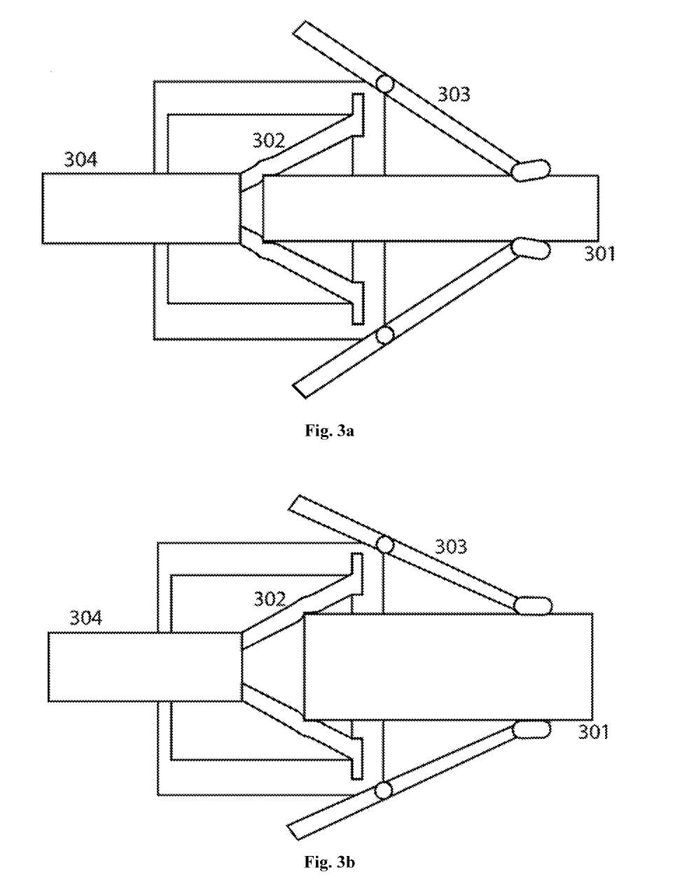

In some embodiments, the stethoscope stem (301) is pressed tight against a tapered deformable membrane (302). This forms an airtight seal around the stem, with an aperture that allows sound waves to travel through into the internal audio channel tubing (304). The stethoscope stem is held rigidly in place by external stabilizers (303). Thus, there is no need for modular design or exchange of components in these embodiments, since the tapered membrane and stabilization arms are easily adjusted to accommodate narrow (FIG. 3a) or broad (FIG. 3b) stethoscope stems.

The adapter is not limited to the embodiments described above. In some embodiments, the sleeve is tapered smoothly or in stepwise fashion to accommodate various stethoscope sizes. In other embodiments, the sleeve is tightened onto the stethoscope stem with a threaded nut and bolt, a compression fitting, a spring-loaded clamp, or any other mechanism. In other embodiments, the sleeve does not exist as a separate component, but is an extension of the internal audio channel. In other embodiments, the stethoscope stem attachment mechanism varies, but a pathway always exists for sound waves to travel through from the stethoscope stem into the internal audio channel.

The internal audio channel (104) leads to the head of the microphone. In the preferred embodiment, this microphone is a miniature, low cost, electret microphone, but the microphone may be any device that transduces mechanical sound waves into electrical signals, such a MEMS microphone. In the preferred embodiment, sound passing through the internal audio channel will be funneled into the microphone and converted into an electrical signal. The signal will pass from the microphone via wire to a printed circuit board (106), which also includes various additional circuit elements. In some embodiments, the signal can be amplified, filtered, and otherwise processed on the board itself; however, in the preferred embodiment, signal processing on the circuit board is minimal. This further reduces power consumption, lowers cost, and increases the battery life of the device. The electrical signal, an analog representation of sound from the patient, is converted into a digital signal and broadcast wirelessly via a wireless transceiver (107). The wireless transmission can take place using any wireless technology, but in the preferred embodiment it is transmitted via Bluetooth.

In the preferred embodiment, the printed circuit board may contain at least one additional element, such as a set of batteries (108) that powers the microphone, board, and transceiver. In the preferred embodiment, these batteries are rechargeable via a wireless qi charger circuit (i.e. electromagnetic induction). In the preferred embodiment, wireless charging eliminates the need for external ports thereby making the device completely sealed and easily cleanable using a variety of disinfection techniques. In some embodiments, additional ports such as an audio jack, power port (109), power switch, volume adjustment, and wireless pairing button may pass through the device's housing in order to be externally accessible. In the preferred embodiment, the device housing itself (110) may be made of plastic or metal and may include insulation to shield it from ambient noise. In the preferred embodiment, the housing is sealed except during assembly and maintenance.

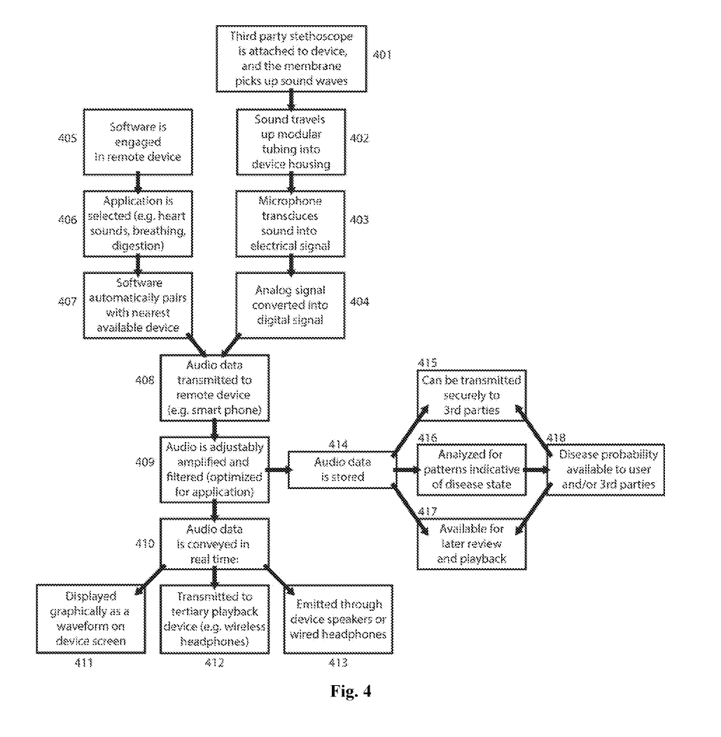

A flow chart illustrating the invention's methodology is shown in FIG. 4 for the preferred embodiment. After sound is detected at a stethoscope's membrane (401), it is transmitted through modular tubing (402) to a microphone, where it is transduced (403) into electrical signals that are converted into a digital format (404). Meanwhile, the user opens requisite software on a remote receiving device (405) and selects the category of sounds (406) they are listening for such as heart sounds, digestive sounds, breathing, etc. In the preferred embodiment, the software will then automatically pair with the nearest device that transmits signals in the requisite format (407), distinguishing between candidates based on the strength of signal perceived or distance between the pairing devices or other methods. The signal containing audio information is then transmitted to a receiving device, which in the preferred embodiment will be conducted over a Bluetooth wireless connection (408). In the preferred embodiment, the receiving device is typically a phone, but the receiving device may also be a computer, speaker, modem, or any other electronic device.

In the preferred embodiment, once the audio data is received, the remote device will filter, amplify, and otherwise process the signal (409), including but not limited to low-pass filtration and/or dynamic amplification steps. The user can adjust the filtration and amplification, and in the preferred embodiment, the default settings are preset to optimally hear sounds of the type previously selected. The audio signal can then be conveyed (410), in real-time or close to real-time, via a variety of methods. The sound waves can be graphically displayed on the screen of the remote device (411), and/or emitted from headphones or a speaker attached directly to the remote device (412), and/or transmitted to a tertiary playback device such as wireless headphones and then emitted (413). In the preferred embodiment, the entire system functions in real-time or close to real-time such that there is negligible delay from the time the sound is produced to the time it is played back through a sound producing device (such as a speaker or headphones). The delay considered negligible depends highly on the end user and the eventual application of the device and can vary from a few milliseconds up to tens of seconds. However, in the preferred embodiment, the total delay will be less than 1 second.

In the preferred embodiment, the user will also have the option of recording and saving audio (414) both in its raw form and post digital signal processing form. In the preferred embodiment, this signal can be securely transmitted (415) to a healthcare professional, a medical institution, and/or any other third party with the requisite permissions, potentially saving time and expenses compared to conventional in-person examinations. In the preferred embodiment, the signal is also analyzed (416) via algorithm, machine learning, and/or direct comparison to stored audio clips. The software can then display possible disease states along with associated probabilities, based on the strength of observed trends and historical false diagnosis rates. The results of this analysis along with the stored audio clips themselves will be available for review and playback by the user and/or third parties (417-418).

In the preferred embodiment, patient rooms in clinics and hospitals can have separate wireless stethoscopes such that the wireless stethoscope device automatically pairs with the phone of the medical staff member who picks up the stethoscope. By enabling the wireless stethoscope component to be easily cleaned between patient visits, the risk of contamination is minimized. Several options are available for sterilization of the invention, which is critical to avoid transmission of diseases between patients. The device has low surface area from its small size, the device transmits signals wirelessly, and its internal components are completely sealed from external contamination. As a result, the device can be cleaned simply by the application of alcohol or other sterilization agent to the stethoscope head, stem, and/or the device's outer housing. FIG. 4 illustrates a sterilization station in the preferred embodiment. This station consists of an ultraviolet light-absorbing outer housing (502), into which the invention (501) coupled or decoupled with the stethoscope head is placed. In the preferred embodiment, this station could also serve to simultaneously charge the invention either via a wired connection (503) or wirelessly by electromagnetic induction. If a device is sensed either via a wired connection or other means once the lid (504) is closed, an ultraviolet light (505) will be automatically activated. Ultraviolet light has been proven to destroy microbial contaminants, and the light will be kept on long enough to ensure a high probability of complete sterilization. In the preferred embodiment, an external display (506) will count down until the required time for sterilization has been achieved, after which the UV light will turn off and the screen readout will indicate that sterilization is complete. In the preferred embodiment, the inside of the sterilization station will be lined with a UV-reflective material, ensuring that UV light reaches all external surfaces of the invention. If the lid is opened prematurely, then the UV light will automatically turn off.

The invention has been described in the above passages and illustrations, but it is understood that this information presents only a preferred embodiment and some other embodiments; it is not intended to restrict the scope or essence of the invention. The concepts, features, and illustrations described herein are not intended to be limiting, and are subject to recombination, alteration and expansion of function and form. A practitioner of ordinary skill in the art will recognize that the embodiments, implementations, and examples described in this specification and shown with reference to the various figures, are all only exemplary and not limiting. There are alternative methods of accomplishing many of the elements, features, and functions that all fall within the spirit and scope of this invention.

What we claim is:

1. A system for converting a sound conductor, wherein the source and sink have a physical connection between them, into a wireless audio transmitter, wherein the source and sink do not need a physical connection between them to provide the function of the system, comprising:
a housing with at least one aperture that creates an acoustic pathway to an acoustic membrane or casing consisting of a tympanic membrane
with the housing also comprising at least one acoustic receiving unit located along the acoustic pathway entering the housing and configured to convert mechanical sound waves to electrical signals
with the housing also comprising at least one circuit configured to wirelessly transmit data from the acoustic receiving unit to a remote secondary device configured to be capable of playing back the audio and/or transmitting the signal to a tertiary device configured to play back the audio

2. The system of claim 1, wherein the tympanic membrane is part of a passive stethoscope chest piece

3. The system of claim 2, wherein an adapter is used to reversibly connect the aperture of the housing and the stethoscope chest piece along the acoustic pathway of sound

4. The system of claim 3, wherein the adapter can change size to fit different sized acoustic conduits exiting the stethoscope chest piece

5. The system of claim 4, wherein the adapter changes size using a compression-based attachment mechanism

6. The system of claim 4, wherein the adapter changes size using a magnetic-based attachment mechanism

7. The system of claim 3, wherein adapters of different sizes can fit different sized acoustic conduits exiting the stethoscope chest piece

8. The system of claim 3, wherein the cross-sectional area of the adapter changes along its body to accommodate different sized acoustic conduits exiting the stethoscope chest piece

9. The system of claim 1, wherein the data is transmitted wirelessly using any available wireless protocol with a range greater than 50 centimeters.

10. The system of claim 1, wherein a seal made of a sound reducing material is placed inside and/or outside the housing to prevent sound dissipation and reduce ambient noise

11. The system of claim 1, wherein the system contains a battery that is rechargeable wirelessly to minimize breaches in the housing of claim 1 to allow for seamless cleaning

12. The system of claim 1, wherein the system contains a battery that is rechargeable via a wire

13. The system of claim 1, wherein the audio extracted from the acoustic receiving unit is amplified, filtered, and/or in any way modified using a circuit in any device receiving the audio data

14. The system of claim 1, wherein the audio extracted from the acoustic receiving unit is amplified, filtered, and/or in any way modified using software in any device receiving the audio data

15. The system of claim 14, wherein the device receiving the audio data does not have a physical connection with the acoustic receiving unit of claim 1

16. The system of claim 1, wherein the audio extracted from the acoustic receiving unit is displayed visually in real-time or close to real-time

17. The system of claim 1, wherein recording, processing, and playback of audio extracted from the acoustic receiving unit is done in real-time or close to real-time

18. A method for converting a passive stethoscope, wherein the source and sink have a physical connection between them, into a wireless stethoscope, wherein the source and sink do not need a physical connection between them to provide the function of a stethoscope, comprising:
converting mechanical sound waves into electrical signals using a microphone
sending audio from the microphone wirelessly to a remote secondary device using a circuit
playing back the audio received from the microphone from the secondary device and/or transmitting the audio from the secondary device to a tertiary device to play back the audio, in real time or close to real time

19. The method of claim 18 further comprising transmitting data wirelessly using any available wireless protocol with a range greater than 50 centimeters

20. The method of claim 18 further comprising amplifying, filtering, and/or in any way modifying the analog audio extracted from the acoustic receiving unit using a circuit in any device receiving the audio data

21. The method of claim 18 further comprising amplifying, filtering, and/or in any way modifying the audio extracted from the acoustic receiving unit using software in any device receiving the audio data

22. The method of claim 18 further comprising displaying the audio extracted from the acoustic receiving unit visually in real-time or close to real-time

23. The method of claim 18 further comprising recording, processing and, playing back the audio extracted from the acoustic receiving unit in real-time or close to real-time

24. The method of claim 18 further comprising analyzing the audio extracted from the acoustic receiving unit to detect specific patterns against a database
